(19) United States
(12) Patent Application Publication
(10) Pub. No.: US 2025/0087365 A1
(43) Pub. Date: Mar. 13, 2025

(54) Communicating Narrative Concerns Entered by Registered Nurses (Concern) Clinical Decision Support and Predictive Modeling Systems and Methods

(71) Applicant: The Trustees of Columbia University in the City of NewYork, New York, NY (US)

(72) Inventors: Sarah Collins ROSSETTI, New York, NY (US); Kenrick CATO, New York, NY (US); Christopher KNAPLUND, New York, NY (US)

(73) Assignee: The Trustees of Columbia University in the City of NewYork, New York, NY (US)

(21) Appl. No.: 18/814,823

(22) Filed: Aug. 26, 2024

Related U.S. Application Data
(60) Provisional application No. 63/555,628, filed on Feb. 20, 2024.
(60) Provisional application No. 63/538,164, filed on Sep. 13, 2023.
(60) Provisional application No. 63/573,041, filed on Apr. 2, 2024.

(51) Int. Cl. G16H 50/30; G16H 50/20; G16H 50/70

(52) U.S. Cl. CPC G16H 50/30; G16H 50/20; G16H 50/70

(57) ABSTRACT

Disclosed are implementations, including a method for medical/clinical monitoring that includes obtaining clinical and physiological measurement data for a patient, and contextual information associated with the patient and representative of location and time at which the clinical and physiological measurement data were obtained, and obtaining clinician behavior data for the clinicians while treating the patient. The method further includes determining intermittently, using an ensemble learning process, based on the measurement data and the contextual information, one of a plurality of medical state prediction models best suited for a current clinical situation associated with the patient. The method additionally includes determining, by the determined medical state prediction model, based on the measurement data and the clinician behavior data, prediction output data representing a medical and/or clinical state trajectory for the patient, and providing notification output data representative of the medical and/or clinical state trajectory for the patient.

### CROSS-REFERENCE TO RELATED APPLICATIONS

This application claims priority to, and the benefit of, U.S. Provisional Application No. 63/538,164, entitled “Communicating Narrative Concerns Entered by Registered Nurses (CONCERN) Clinical Decision Support and Predictive Modeling System” and filed Sep. 13, 2023, U.S. Provisional Application No. 63/555,628, entitled “Communicating Narrative Concerns Entered by Registered Nurses (CONCERN) Clinical Decision Support and Predictive Modeling System” and filed Feb. 20, 2024, and U.S. Provisional Application No. 63/573,041, entitled “Communicating Narrative Concerns Entered by Registered Nurses (CONCERN) Clinical Decision Support and Predictive Modeling System” and filed Apr. 2, 2024, the contents of all of which are incorporated herein by reference in their entireties.

### STATEMENT REGARDING FEDERALLY SPONSORED RESEARCH

This invention was made with government support under NR016941 awarded by the National Institutes of Health (NIH). The government has certain rights in the invention.

### BACKGROUND

Clinical and physiological measurement data obtained for a patient can provide useful information about the instantaneous medical state/condition of the patient. However, those measurements have limited predictive power for longer term tracking of the patient's medical state, and for achieving early detection of deterioration of the patient.

### SUMMARY

In some variations, a method for medical monitoring is provided that includes obtaining (e.g., directly from medical sensor devices, or from a data repository collecting and managing data entered by clinicians or data received from other sources, or directly through manual data entry) clinical and physiological measurement data for a patient, and contextual information associated with the patient and representative of location and time at which the clinical and physiological measurement data were obtained, and obtaining clinician behavior data representative of behavior and activity of one or more clinicians while medically treating the patient. The method further includes determining intermittently, using an ensemble-based machine learning process, based on at least a subset of the clinical and physiological measurement data for the patient and the contextual information associated with the patient, one of a plurality of medical state prediction models best suited for a current clinical situation associated with characteristics of the patient (e.g., as may be determined based on the measurement data and the contextual information, and/or based on the clinician behavior data), with the plurality of medical state prediction models being implemented on one or more machine learning systems. Having determined (selected) the suitable model to use (e.g., from the plurality of models optimized for specific patient circumstances), the method further includes determining, by the determined one of the plurality of medical state prediction models, based on the clinical and physiological measurement data for the patient and the clinician behavior data (which may be provided as metadata of the clinical and psychological data), prediction output data representing a medical and/or clinical state trajectory for the patient. The method additionally includes providing notification output data representative of the medical and/or clinical state trajectory for the patient.

In some variations, a medical monitoring system is provided that includes a communication unit to obtain clinical and physiological measurement data for a patient, and contextual information associated with the patient and representative of location and time at which the clinical and physiological measurement data were obtained, and to obtain (e.g., from electronic health records, or EHR) clinician behavior data representative of behavior and activity of one or more clinicians while medically treating the patient. The system further includes one or more memory storage devices and one or more processors in electrical communication with the one or more memory storage devices and the communication unit. The one or more processors are configured to determine intermittently, using an ensemble learning process, based on at least a subset of the clinical and physiological measurement data for the patient and the contextual information associated with the patient, one of a plurality of medical state prediction models best suited for a current clinical situation associated with characteristics of the patient, with the plurality of medical state prediction models being implemented on one or more machine learning systems. The one or more processors are further configured to determine, by the determined one of the plurality of medical state prediction models, based on the clinical and physiological measurement data for the patient and the clinician behavior data, prediction output data representing a medical and/or clinical state trajectory for the patient, and provide notification output data representative of the medical and/or clinical state trajectory for the patient.

In some variations, a non-transitory computer readable media is provided that includes computer instructions executable on a processor-based device to obtain clinical and physiological measurement data for a patient, and contextual information associated with the patient and representative of location and time at which the clinical and physiological measurement data were obtained, and obtain clinician behavior data representative of behavior and activity of one or more clinicians while medically treating the patient. The computer instructions further cause the processor-based device to determine intermittently, using an ensemble learning process, based on at least a subset of the clinical and physiological measurement data for the patient and the contextual information associated with the patient, one of a plurality of medical state prediction models best suited for a current clinical situation associated with characteristics of the patient, with the plurality of medical state prediction models implemented on one or more machine learning systems. The computer instructions further cause the processor-based device to determine, by the determined one of the plurality of medical state prediction models, based on the clinical and physiological measurement data for the patient and the clinician behavior data, prediction output data representing a medical and/or clinical state trajectory for the patient, and provide notification output data representative of the medical and/or clinical state trajectory for the patient.

Embodiments of the method, system, and the computer readable media may include at least some of the features described in the present disclosure. Other features and advantages of the invention are apparent from the following description, and from the claims.

### BRIEF DESCRIPTION OF THE DRAWINGS

These and other aspects will now be described in detail with reference to the following drawings.

FIG. 1 is a block diagram of an example medical monitoring system to track clinical status and predict medical trajectories for one or more patients.

FIG. 2A is an example output display produced by a user interface to present a patient's medical risk information.

FIG. 2B is an example of an EHR's patient list screen rendered on the user interface.

FIG. 3 is a flowchart of an example medical monitoring procedure.

Like reference symbols in the various drawings indicate like elements.

### DESCRIPTION

The proposed framework described herein is used to model clinicians' (e.g., nurses', technicians', physicians', nurse practitioners', physician assistants', etc.) concern for patient medical state (e.g., whether the patient's medical condition is deteriorating). Under the proposed approaches and solutions implemented, the framework leverages the expertise of the clinicians (e.g., nurses) in understanding contextual information that modeling just based on patient physiological changes cannot. During testing and evaluation of the proposed framework, the racial bias of the CONCERN CDS was compared to other predictive scores like the National Early Warning Score. The testing and evaluation showed no statistically significant differences indicating racial bias. External validation of the model used by the proposed framework was also performed, including in a multi-site clinical trial. The proposed approaches may also be used to monitor patient acuity on a unit and required clinical staffing, and to otherwise manage resource usage (e.g., ventilators, dialysis).

Nursing surveillance is a core part of nursing practice whereby nurses provide a purposeful and ongoing acquisition, interpretation, and synthesis of patient data for clinical decision-making with the goal of predicting and preventing adverse events in individual patients. In doing so, nurses recognize subtle, yet observable, clinical changes that are rarely captured in physiological data and not well-displayed in electronic health records (EHR's), such as pallor change with incremental increased need for supplemental oxygen, slower recovery of the patient's arterial blood pressure after the patient is turned, or subtle changes in mental status from baseline. At each assessment point, these changes may not be significant enough to require immediate intervention or intensive care unit (ICU) transfer but are routinely observed and documented by nurses. Nurses opt to record extra data points and narrative comments in the EHR to visually link clinical observations and highlight changes in a patient's condition, but these documentation practices are not evident to prescribing providers. A key indicator of a nurse's concern for increased risk of patient deterioration is increased surveillance, which is a valid and the most frequent reason for initiation of a rapid response. When there is a lack of shared team situational awareness (insufficient identification, comprehension, and projection of future risk shared among the care team) medical interventions are delayed.

The proposed framework is the Communicating Narrative Concerns Entered by Registered Nurses (CONCERN) Clinical Decision Support System (CDS). The framework uses the Healthcare Process Modeling approach to phenotype clinician behaviors for exploiting the Signal Gain of Clinical Expertise (HPM-ExpertSignals) modeling technique that phenotypes clinician behaviors as proxies of clinician knowledge and expertise to quantify patient or clinician properties that are not directly measurable. These interactions are measured as explicit concern signals (i.e., content of recorded data) and implicit concern signals (i.e., interaction patterns) in clinical information systems. The HPM-ExpertSignals are used to inform predictive models.

HPM-ExpertSignals is used to predict, (1) clinician concern for change in patient states (e.g., deterioration, patient readiness for discharge), (2) recourses usage (e.g., bed-tracking, ventilator tracking, dialysis usage), (3) phenotyping nurses' decision-making based on documentation practices, and (4) learning clinician behavior for evidence-based practice in a specific setting to improve care for application across settings.

The proposed approach uses EHR metadata (e.g., date/time stamps and data type) of nursing surveillance activities to identify deterioration up to 42 hours earlier than EWS that use physiological indicators. The EHR metadata can therefore be used in clinical decision support to make the care team aware so that more timely interventions can be performed. The proposed approach differs from other EWS in a couple of important ways. For example, the proposed approach models a clinician's activity to predict patient deterioration. Additionally, by using an ensemble modelling method, crucial time dependent signals are leveraged by modeling, for instance, every hour of the day. Using this novel approach that includes relying on metadata patterns (recorded clinical or physiological values can also be incorporated into model), the model continuously monitors nurses' concern levels that reflect nurses increased surveillance, such as, for example, 1) increased frequency of assessments (e.g., respiratory rate checked every 2 hours for an acute care floor patient), 2) assessments being done at uncommon times (e.g., checking vital signs in the middle of the night for an acute care floor patient), and/or 3) nursing medication administration interventions such as not administering a scheduled medication (typically due to unstable clinical conditions). Other factors, measurements, and observations that reflect practitioners' (nurses') level of concern may also be relied upon in the proposed approach.

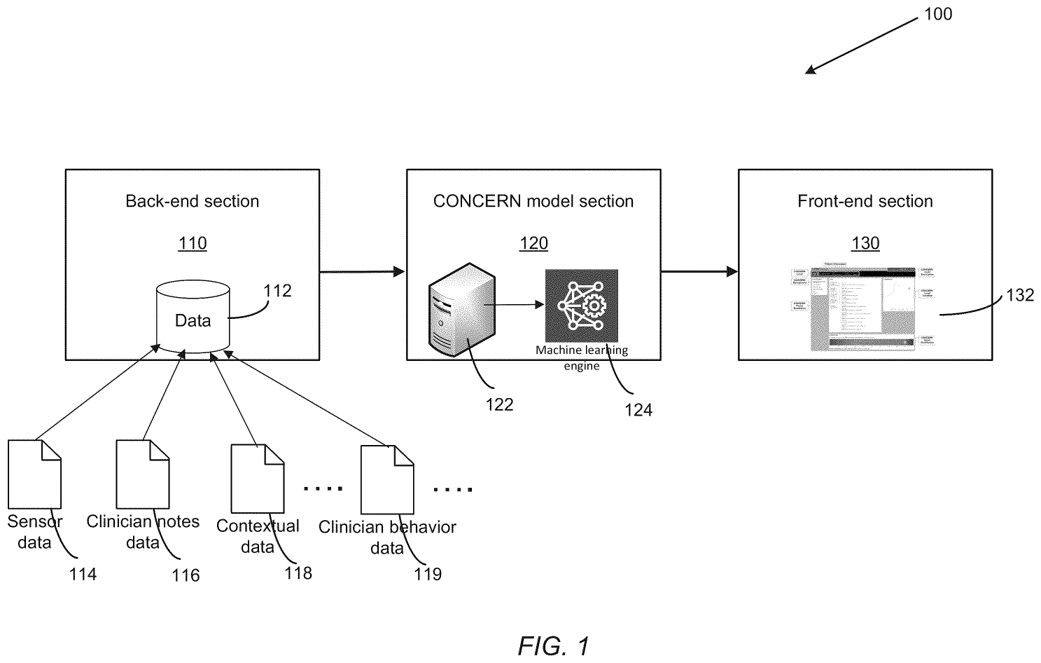

As depicted in FIG. 1, showing a block diagram 100 of an example framework to track clinical status of one or more patients, the CONCERN CDS is made up of three parts. First, the CONCERN framework includes a back-end engine 110 that controls the reading of patient-related data, and the writing of the predictive CONCERN score to a repository. In the example illustrated in FIG. 1, a data repository 110 is configured to receive, manage and maintain the data, and provide processed data to downstream processes. The data repository may receive data from such diverse sources as sensors collecting data about patients (provided by sensor data records 114, clinician notes about the patients (e.g., medical chart data, clinician observations, and so on) provided by the clinician notes data records 116, contextual data 118 (e.g., location and time at which measurements were taken), clinician behavior data 119 (representative of behavior and activity of one or more clinicians while medically treating the patient), historical data about the patients (not shown), etc. The clinician behavior data 119 can be determined from analysis of data entries by the clinician and their context (e.g., based on algorithms, or using a machine learning system to identify unusual activity that may be indicative of some medical change in the status of the patient). The clinician behavior can also be determined based on other sources of data captures, such as video data that can be analyzed (through algorithmic processes or machine learning models), and data sources that indirectly point to unusual patterns of behavior and/or interactions with a patient by the clinician(s). The data repository may be controlled by one or more computing devices such as a server 122 depicted in FIG. 1, or by a dedicated data management engine (not shown). The back-end section's data management functionality may include performing various pre-processing operations on received data such as, for example, arranging incoming input data into uniformly formatted data records, removing suspicious or errant data, normalizing data into pre-defined scales or into pre-defined discrete values, etc.

A second part of the CONCERN CDS system includes a CONCERN model section 120, which may include one or more processor-based systems such as the server 122, that are configured to implement machine learning models executing on a machine learning engine 124 (the machine learning engine may alternatively be implemented on a dedicated processor-based module different than server 122). The CONCERN model processes data (e.g., received from the repository 112), or to process patient-related data (including nurses, or other clinician, notes, and data records representing various actions taken with respect to patients) uses the patient data to generate a score (e.g., using a selected machine learning model that best fits the circumstance of the patient being monitored) to predict the patient's likelihood of clinical deterioration within some selected (or determined) period of time (e.g., in the next 24 hours).

The third part of the framework is a front end section 130, that includes a clinician, patient, or caregiver facing application 132 (e.g., a user input-output interface) that is implemented for, and executed on, one or more processor-based systems (such as the server 122) to view the CONCERN predictions and other germane data. As will be discussed below in greater detail, the front-end section 130 also presents via the user interface application 132 what are the factors that are driving that prediction, a trend of the patient's CONCERN level over time, and where that patient's CONCERN score fits in relation to all of the other patients' scores. The CONCERN framework has a greater lead time than other EWS's systems and can include bias mitigation approaches to support equitable care.

The risk prediction model (which, as noted, may be implemented on a machine learning engine such as the engine 124) of the proposed framework is nonlinear, with time varying variables (e.g., vital signs recorded at variable times within a clinical shift) which requires accuracy evaluation that must take many aspects of the model, data, and clinical problem into account. In some embodiments, the proposed CONCERN EWS framework processes, at regular or irregular time intervals (e.g., every hour), EHR data from the past 24 hours through a set of ensemble models (selecting one that best fits the patient characteristics), and calculates a risk score (green, yellow, or red).

CONCERN EWS' conceptual modeling approach and model factors can be summarized as reflecting patterns of increased nurses' surveillance and the resultant nursing interventions consistent with an observed change in the patient's clinical state. Increased nurses' surveillance in the model may be measured, for example, when a nurse assesses vital signs every two (2) hours for a specific patient displaying subtle changes even though the clinical unit policy only requires assessment every six (6) hours. As an example of a nursing intervention consistent with observed but subtle changes in the patient's clinical state, a nurse may decide not to administer a scheduled medication, such as metoprolol, because the patient's heart rate is hovering around 60 beats per minute and their usual baseline is around 90 beats per minute. While medications are ordered by a prescribing provider in the hospital setting, the administration is completed by a nurse and requires the nurse to assess the appropriateness of that medication given the patient's condition prior to administration. The approach by the proposed framework also considers if nursing assessments or interventions are performed at non-common times, based on data-driven analyses. The CONCERN EWS can include a monitoring function to check that all variables are available to the model and that the volume of data for each variable was consistent with historical volumes, with automated emailed notifications of issues. The CONCERN EWS may also implement robust error reporting and logging functionalities for each step in the data pipeline, including for data inputs, pre-processing, prediction generation, and CONCERN score writing to the EHR. A system performance error can be deemed to occur, in one example, when the automated prediction score is not being generated every hour for eligible patients. During experimentation and evaluation of the proposed framework, it was determined that the few times that automated predictions did not occur were due to a web service error. The error reporting implementation notifies appropriate personnel of the error that has occurred, who can then remedy the issue (e.g., manually triggering the prediction processes for all patients).

Through experimentation on retrospective data sets of hospitalized patients' encounters in the EHR, a machine learning modeling approach was developed and refined to focus on temporal patterns of nursing observations, using, for example, ensemble learning to identify the best performing model (from a set of models) for a patient's specific clinical situation. For instance, ensemble learning facilitates the identification of the best model for a patient who has been in an ICU for, e.g., 5 hours on a Friday night (a different model may be selected for the same patient if a different set of circumstances presented themselves, e.g., if the patient has been in the ICU for 5 hours during the middle of week), and to make the early warning system (EWS) transparent and explainable. Selection of the appropriate model allows the framework to predict more accurately clinical deterioration within some time horizon (e.g., within the next 24 hours). For model building, patient deterioration is defined as the first occurrence of unanticipated transfer to the ICU, rapid response transfer, mortality, or cardiopulmonary arrest. Thus, the proposed approach, including the use of ensemble models in the CONCERN framework facilitates time-dependent modeling that achieves to implement a more effective and accurate early warning system

In some embodiments, every hour (or some other time interval of greater or lower temporal resolution) the CONCERN EWS processes electronic health records (EHR) data from, for example, the past 24 hours (or some longer or shorter preceding period) through the ensemble models that best fit the patient characteristics, calculating a risk score. The proposed framework can thus realize a decision support implementation of EWSs that use a similar multi-factorial, advanced computational, and explainable modeling approach. In some examples, other machine learning predictive models, implemented on various types of machine learning architectures, may be used to generate prediction output data representing a medical and/or clinical state trajectory for a patient.

The CONCERN EWS exchanges data and integrates into the EHR using, in some embodiments, the FHIR (Fast Healthcare Interoperability Resources) standard for exchanging healthcare information electronically. As noted, the CONCERN framework also includes a user interface (such as the interface 132, implemented as part of the front-end section 130 using a shared or dedicated processor-based system) to present relevant output data, be it in visual and/or audible form, that provides data pertaining to the monitored patient(s) and/or medical trajectory (e.g., evolving medical condition risk) to the patient(s)).

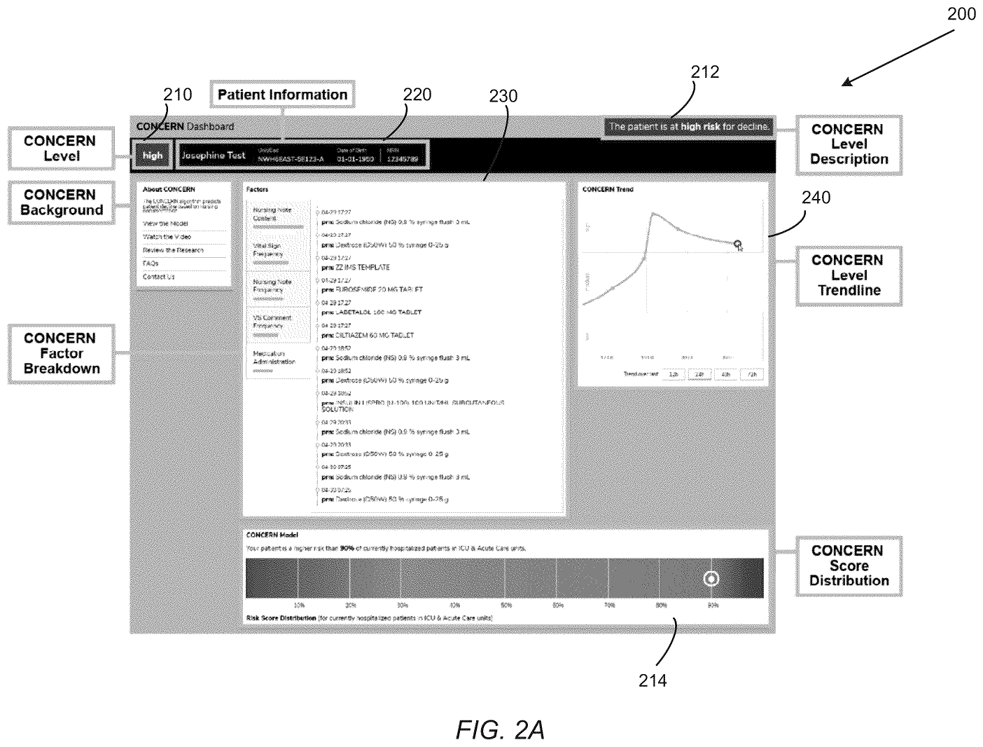

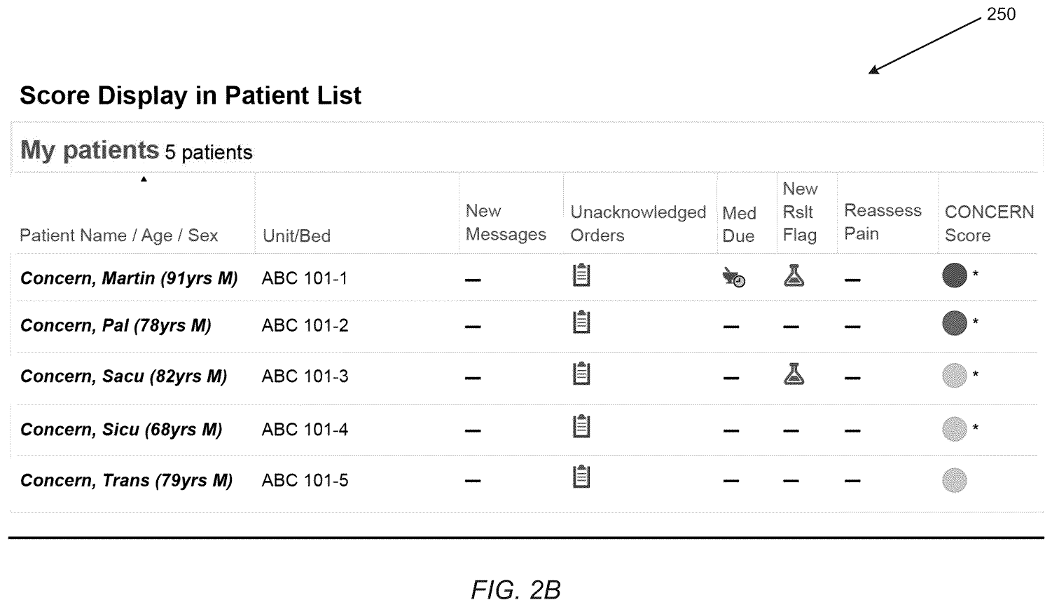

An example output display produced by the user interface is shown in FIG. 2A, providing a screen shot of a dashboard 200 displaying output for a particular patient. In some embodiments, the user interface dashboard 200 may be invoked in response to, for example, selecting a patient from a clinician's EHR's patient list (e.g., through double clicking on visual output display, or through use of some other methodology of providing user input). An example of an EHR's patient list 250, rendered on a user output interface, is shown in FIG. 2B (it is noted that the patient list 250 may be rendered on a same screen on which a patient's detailed prediction screen, such as dashboard 200, is displayed). The list of patients may be displayed alongside respective generated risk score output indicators such as, for example, a graphical element display, such as a geometric shape like a circle, square, etc.), a customized icon, a color scale of green, yellow, red, or any other type of output indicator. In some embodiments, the risk level indicated for the listed patients may be in the form of a numerical risk level, a verbal risk indicator, and so on.

The EHR's patient list and risk score indicator links to the screen of the dashboard 200 illustrated in FIG. 2A, which includes detailed information about the specific prediction for the selected patient. The dashboard 200 can include, for example, a concern level indicator 210 (depicted in the example dashboard 200 in the top left corner) which typically displays the risk level information that was presented on the clinician EHR's patient list. Thus, the concern level indicator (here a square) includes a color filling indicative of the risk level to the particular patient (e.g., a red filling) with the wording “high,” indicating a high risk prediction for the patient medical trajectory given the existing input factors that feed into the machine learning system comprising the CONCERN ESW. The dashboard may also include a “concern level description” field 212 providing a more explicit description of the nature of the risk level indication provided by the concern level indicator 210, as well a graphic representation of the spectrum of risk levels (e.g., rendered in the “CONCERN model” display region 214) to graphically illustrate the gravity of the prediction on the spectrum of risk. The CONCERN model display region can provide the patient's risk severity either relative to other currently hospitalized patients, which can assist with triaging decisions and resource allocation, or relative to a general population of patients (locally, or in other hospitals) over some period of time. The “CONCERN model” display area can also provide a more refined grading of the patient's risk level (e.g., to illustrate whether the current “High” risk level is at the top of the scale, or whether it has not approached the most critical level of the spectrum). In the example of FIG. 2A, the “High” risk level is indicated to be on the boundary between the second to highest severity area, and the highest severity area.

Positioned next to the indicator 210 is a “patient information” output area 220 providing details about the selected patient. The dashboard 200 additionally includes a factor's area 230 detailing the factors that determined a patient's score. In the embodiments of FIG. 2A, users (such as the clinician seeking details about a particular selected patient) can also click on the factors that determined the score to view the clinical data associated with that factor. The factors include one or more granular CONCERN model features, e.g., vital sign frequency factor includes measurement patterns related to each type of vital sign, nurses' chart entries for the patient, nurses' actions for the patients, and the timing information (such as frequency) associated with those actions, etc. As also seen in FIG. 2A, the dashboard 200 can also include a “CONCERN trend” display area 240 that provides a graphical representation of the CONCERN (e.g., risk) trend line, to give the clinician an easily understood progression of the predicted risk of the patient over some period of time, such as over the preceding 72 hours, thus capturing an improvement or a worsening of the patient's predicted risk trajectory.

A multi-site clinical trial showed statistically and clinically significant decrease on patient mortality and sepsis for patients randomized to the intervention group versus those in a control group. There were also statistically significant impacts on length of stay and unanticipated transfer to ICU for patients randomized to the intervention group versus the control group.

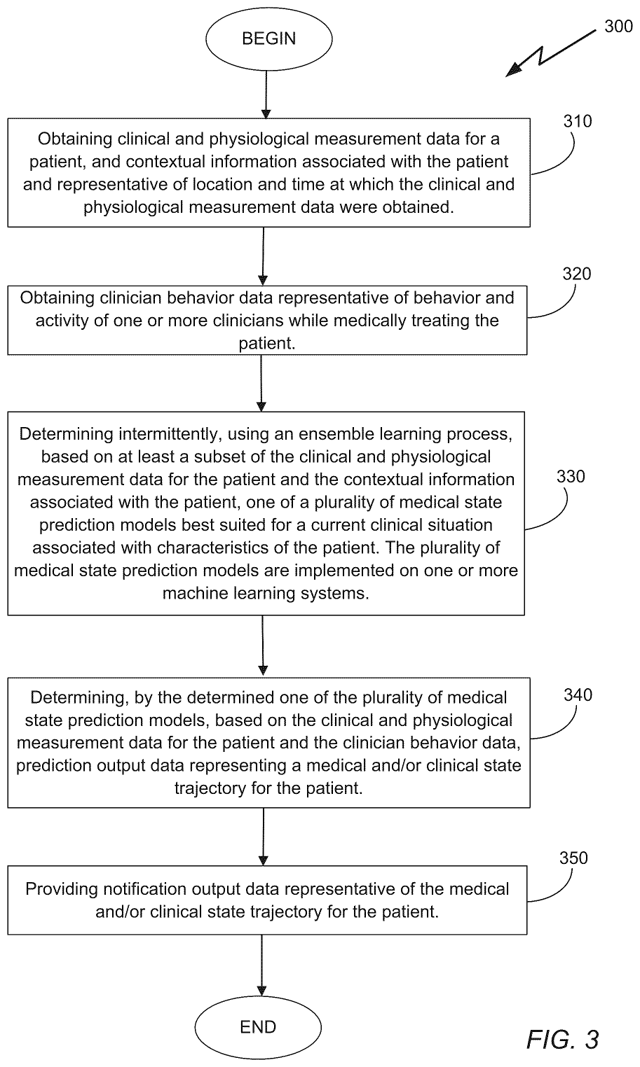

With reference next to FIG. 3, a flowchart of an example procedure 300 for medical monitoring is shown. The procedure 300 includes obtaining 310 (e.g., directly from medical sensor devices, or from a repository to store and management data) clinical and physiological measurement data for a patient, and contextual information associated with the patient and representative of location and time at which the clinical and physiological measurement data were obtained, and obtaining 320 clinician behavior data representative of behavior and activity of one or more clinicians while medically treating the patient. In some examples, the contextual information may include treatment place (e.g., ICU, ER) at which medical treatment is being administered to the patient, and length of stay of the patient at the treatment place. The clinician behavior data representative of the behavior and the activity of the one or more clinicians while treating the patient may include one or more of, for example, frequency of surveillance by the one or more clinicians of the patient, interaction patterns between the one or more clinicians and the patient, type and frequency of interventions (e.g., turning the patient over to prevent pressure ulcers, increasing oxygen supply, checking on the patient, administering medications, etc.) performed by the one or more clinicians for the patient, and/or changes in the patient's vital signs following an intervention.

With continued reference to FIG. 3, the procedure 300 further includes determining 330 intermittently, using an ensemble learning process, based on at least a subset of the clinical and physiological measurement data for the patient and the contextual information associated with the patient, one of a plurality of medical state prediction models best suited for a current clinical situation associated with characteristics of the patient, with the plurality of medical state prediction models being implemented on one or more machine learning systems. In various examples, determining intermittently the one of the plurality of medical state prediction models may include applying at regular or irregular intervals the ensemble learning process to select at a beginning of each of the intervals, based on a state of the at least the subset of clinical and physiological measurement data and the contextual information associated with the patient at the beginning of the each of the intervals, the one of the plurality of medical state prediction models. In such embodiments, determining the prediction output data representing the medical and/or clinical state trajectory for the patient may include re-processing, at the beginning of each of the intervals using the respective one of the plurality of the medical state prediction models, at least a portion of the clinical and physiological measurement data for the patient and the clinician behavior data accumulated during a specified period preceding the beginning of each of the intervals, to determine the prediction output data representing the medical and/or clinical state trajectory for the patient during a current interval. In various examples, determining the one of the plurality of medical state prediction models can include determining based further on the clinician behavior data the one of the plurality of medical state prediction models.

Having determined (selected) the suitable model to use (e.g., from a plurality of models optimized for specific patient circumstances and characteristics), the procedure further includes determining 340, by the determined one of the plurality of medical state prediction models, based on the clinical and physiological measurement data for the patient and the clinician behavior data (which may be provided as metadata of the clinical and physiological data), prediction output data representing a medical and/or clinical state trajectory for the patient. The method additionally includes providing 350 notification output data representative of the medical and/or clinical state trajectory for the patient.

In various examples, providing the notification output data may include providing notification on a user interface of a predicted medical state deterioration of the patient within a specified future time period. Providing the notification of the predicted medical state deterioration can include rendering an indicator of the predicted medical scale on a graphical severity scale to present the patient's risk severity relative to other currently hospitalized patients at a facility where the patient is hospitalized, or relative to a general population of hospitalized patients. In some examples, providing the notification output data can include rendering a list of patients being treated at a facility on the user interface and respective ones of predicted medical state deteriorations determined for the patients, with the list of patients including the patient, and rendering on the user interface, in response to selecting the patient from the list of patients, a dashboard presenting information that includes the respective predicted medical state deterioration for the patient, and one or more of, for example, details of the patient, factors that contributed to the determination of the predicted medical state, a graphic representation of the predicted medical state deterioration of the patient relative to other patients, and/or a trend line graph showing the predict medical state deterioration of the patient over a pre-determined period of time.

The proposed framework was tested and evaluated to produce the following outcomes. Patients whose care team received the CONCERN EWS had a lower probability of dying or having sepsis in the hospital, shorter average LOS (length of stay), and a higher probability of being transferred to the ICU. In-hospital mortality, sepsis, and LOS are important performance indicators. ICU transfer during the early period of deterioration is a “window of critical opportunity” due to its association with improved survival. Early ICU transfers can facilitate timely clinical interventions and alter the trajectory of a patient's clinical progression while there is still an opportunity to prevent adverse outcomes, especially for patients with elevated physiological risks. The testing and evaluation of the proposed framework was achieved in a pragmatic cluster randomized controlled trial for impact on patient outcomes. During that trial, usage data was also captured and sub-analyses of process measures performed.

There are several reasons why the CONCERN EWS has superior predictive power to other EWS's, whose lack of clinical impact may be attributed to insufficient lead time to alter a patient's clinical trajectory and the use of only a subset of patient data, such as physiological values, while ignoring nursing assessment and observational data that is available earlier and is information-rich. The implementation of the CONCERN intervention includes various steps that are not always considered when translating a predictive model to the clinical setting (e.g., end-user input, external validity of findings, model fairness, real-time data availability, and healthcare process effects) and resulted in 42-hour greater lead time than other EWS's. The CONCERN EWS was developed by an interdisciplinary team of clinician informaticians and data scientists to overcome current limitations to EWS's by using nursing assessment and observational data, integrating with existing clinical workflows, and evaluating transparency and explainability (shown to be key for clinician trust). By using nursing surveillance patterns the CONCERN EWS overcomes the limitations of physiological measurements and leverages an already existing expert knowledge base (namely, nurses' pattern recognition and data synthesis skills). For example, previous research identified strong predictive signals of a patient's clinical state using optional nursing documentation (i.e., not required by policy) when nurses take the time to record more observations or notes than mandated for that shift.

Implementing the proposed framework and performing the various techniques and operations described herein may be facilitated by a controller device(s) (e.g., a processor-based computing device). Such a controller device may include a processor-based device such as a computing device, and so forth, that typically includes a central processor unit or a processing core. The device may also include one or more dedicated learning machines (e.g., neural networks) that may be part of the CPU or processing core. In addition to the CPU, the system includes main memory, cache memory and bus interface circuits. The controller device may include a mass storage element, such as a hard drive (solid state hard drive, or other types of hard drive), or flash drive associated with the computer system. The controller device may further include a keyboard, or keypad, or some other user input interface, and a monitor, e.g., an LCD (liquid crystal display) monitor, that may be placed where a user can access them.

The controller device is configured to facilitate, for example, monitoring medical state of a patient based on the patient's physiological data and based on clinician behavior data. In some embodiments, the controller (or a different controller) is configured to monitor behavior of the operation of the system (for the purposes of testing and evaluation), including collecting audit/log files of clinician usage patterns of the system. The storage device may thus include a computer program product that when executed on the controller device (which, as noted, may be a processor-based device) causes the processor-based device to perform operations to facilitate the implementation of procedures and operations described herein. The controller device may further include peripheral devices to enable input/output functionality. Such peripheral devices may include, for example, flash drive (e.g., a removable flash drive), or a network connection (e.g., implemented using a USB port and/or a wireless transceiver), for downloading related content to the connected system. Such peripheral devices may also be used for downloading software containing computer instructions to enable general operation of the respective system/device. Alternatively and/or additionally, in some embodiments, special purpose logic circuitry, e.g., an FPGA (field programmable gate array), an ASIC (application-specific integrated circuit), a DSP processor, a graphics processing unit (GPU), application processing unit (APU), etc., may be used in the implementations of the controller device. Other modules that may be included with the controller device may include a user interface to provide or receive input and output data. The controller device may include an operating system.

In implementations based on learning machines, different types of learning architectures, configurations, and/or implementation approaches may be used. Examples of learning machines include neural networks, including convolutional neural network (CNN), feed-forward neural networks, recurrent neural networks (RNN), etc. Feed-forward networks include one or more layers of nodes (“neurons” or “learning elements”) with connections to one or more portions of the input data. In a feedforward network, the connectivity of the inputs and layers of nodes is such that input data and intermediate data propagate in a forward direction towards the network's output. There are typically no feedback loops or cycles in the configuration/structure of the feed-forward network. Convolutional layers allow a network to efficiently learn features by applying the same learned transformation(s) to subsections of the data. Other examples of learning engine approaches/architectures that may be used include generating an auto-encoder and using a dense layer of the network to correlate with probability for a future event through a support vector machine, constructing a regression or classification neural network model that indicates a specific output from data (based on training reflective of correlation between similar records and the output that is to be identified), etc. Further examples of learning architectures that may be used to implement the framework described herein include language models architectures, large language model (LLM) learning architectures, auto-regressive learning approaches, etc. In some embodiments, encoder-only architectures, decoder-only architectures, encoder-decoder architecture may also be used in implementations of the framework described herein.

The neural networks (and other network configurations and implementations for realizing the various procedures and operations described herein) can be implemented on any computing platform, including computing platforms that include one or more microprocessors, microcontrollers, and/or digital signal processors that provide processing functionality, as well as other computation and control functionality. The computing platform can include one or more CPU's, one or more graphics processing units (GPU's, such as NVIDIA GPU's, which can be programmed according to, for example, a CUDA C platform), and may also include special purpose logic circuitry, e.g., an FPGA (field programmable gate array), an ASIC (application-specific integrated circuit), a DSP processor, an accelerated processing unit (APU), an application processor, customized dedicated circuitry, etc., to implement, at least in part, the processes and functionality for the neural network, processes, and methods described herein. The computing platforms used to implement the neural networks typically also include memory for storing data and software instructions for executing programmed functionality within the device. Generally speaking, a computer accessible storage medium may include any non-transitory storage media accessible by a computer during use to provide instructions and/or data to the computer. For example, a computer accessible storage medium may include storage media such as magnetic or optical disks and semiconductor (solid-state) memories, DRAM, SRAM, etc.

The various learning processes implemented through use of the neural networks described herein may be configured or programmed using TensorFlow (an open-source software library used for machine learning applications such as neural networks). Other programming platforms that can be employed include keras (an open-source neural network library) building blocks, NumPy (an open-source programming library useful for realizing modules to process arrays) building blocks, PyTorch, JAX, and other machine learning frameworks.

Computer programs (also known as programs, software, software applications or code) include machine instructions for a programmable processor, and may be implemented in a high-level procedural and/or object-oriented programming language, and/or in assembly/machine language. As used herein, the term “machine-readable medium” refers to any non-transitory computer program product, apparatus and/or device (e.g., magnetic discs, optical disks, memory, Programmable Logic Devices (PLDs)) used to provide machine instructions and/or data to a programmable processor, including a non-transitory machine-readable medium that receives machine instructions as a machine-readable signal.

In some embodiments, any suitable computer readable media can be used for storing instructions for performing the processes/operations/procedures described herein. For example, in some embodiments computer readable media can be transitory or non-transitory. For example, non-transitory computer readable media can include media such as magnetic media (such as hard disks, floppy disks, etc.), optical media (such as compact discs, digital video discs, Blu-ray discs, etc.), semiconductor media (such as flash memory, electrically programmable read only memory (EPROM), electrically erasable programmable read only Memory (EEPROM), etc.), any suitable media that is not fleeting or not devoid of any semblance of permanence during transmission, and/or any suitable tangible media. As another example, transitory computer readable media can include signals on networks, in wires, conductors, optical fibers, circuits, any suitable media that is fleeting and devoid of any semblance of permanence during transmission, and/or any suitable intangible media.

Although particular embodiments have been disclosed herein in detail, this has been done by way of example for purposes of illustration only, and is not intended to be limiting with respect to the scope of the appended claims, which follow. Features of the disclosed embodiments can be combined, rearranged, etc., within the scope of the invention to produce more embodiments. Some other aspects, advantages, and modifications are considered to be within the scope of the claims provided below. The claims presented are representative of at least some of the embodiments and features disclosed herein. Other unclaimed embodiments and features are also contemplated.

What is claimed is:

1. A method for medical monitoring, the method comprising:
obtaining clinical and physiological measurement data for a patient, and contextual information associated with the patient and representative of location and time at which the clinical and physiological measurement data were obtained;
obtaining clinician behavior data representative of behavior and activity of one or more clinicians while medically treating the patient;
determining intermittently, using an ensemble learning process, based on at least a subset of the clinical and physiological measurement data for the patient and the contextual information associated with the patient, one of a plurality of medical state prediction models best suited for a current clinical situation associated with characteristics of the patient, wherein the plurality of medical state prediction models are implemented on one or more machine learning systems;
determining, by the determined one of the plurality of medical state prediction models, based on the clinical and physiological measurement data for the patient and the clinician behavior data, prediction output data representing a medical and/or clinical state trajectory for the patient; and
providing notification output data representative of the medical and/or clinical state trajectory for the patient.

2. The method of claim 1, wherein the contextual information comprises:
treatment place at which medical treatment is being administered to the patient, and length of stay of the patient at the treatment place.

3. The method of claim 1, wherein determining intermittently the one of the plurality of medical state prediction models comprises:
applying at regular or irregular intervals the ensemble learning process to select at a beginning of each of the intervals, based on a state of the at least the subset of clinical and physiological measurement data and the contextual information associated with the patient at the beginning of the each of the intervals, the one of the plurality of medical state prediction models.

4. The method of claim 3, wherein determining the prediction output data representing the medical and/or clinical state trajectory for the patient comprises:
re-processing, at the beginning of the each of the regular or irregular intervals, using the selected one of the plurality of the medical state prediction models, at least a portion of the clinical and physiological measurement data for the patient and the clinician behavior data accumulated during a specified period preceding the beginning of the each of the intervals, to determine the prediction output data representing the medical and/or clinical state trajectory for the patient during a current interval.

5. The method of claim 1, wherein providing the notification output data comprises:
providing notification on a user interface of a predicted medical state deterioration of the patient within a specified future time period.

6. The method of claim 5, wherein providing the notification of the predicted medical state deterioration comprises:
rendering an indicator of the predicted medical scale on a graphical severity scale to present the patient's risk severity relative to other currently hospitalized patients at a facility where the patient is hospitalized, or relative to a general population of hospitalized patients.

7. The method of claim 1, wherein providing the notification output data comprises:
rendering a list of patients being treated at a facility on the user interface and respective ones of predicted medical state deteriorations determined for the patients, the list of patients includes the patient; and
rendering on the user interface, in response to selecting the patient from the list of patients, a dashboard presenting information that includes the respective predicted medical state deterioration for the patient, and one or more of: details of the patient, factors that contributed to the determination of the predicted medical state, a graphic representation of the predicted medical state deterioration of the patient relative to other patients, and a trend line graph showing the predict medical state deterioration of the patient over a pre-determined period of time.

8. The method of claim 1, wherein the clinician behavior data representative of the behavior and the activity of the one or more clinicians while treating the patient comprises one or more of: frequency of surveillance by the one or more clinicians of the patient, interaction patterns between the one or more clinicians and the patient, type and frequency of clinical interventions performed by the one or more clinicians for the patient, or changes in the patient's vital signs following a clinical intervention.

9. The method of claim 1, wherein determining the one of the plurality of medical state prediction models comprises:
determining based further on the clinician behavior data the one of the plurality of medical state prediction models.

10. A medical monitoring system comprising:
a communication unit to:
obtain clinical and physiological measurement data for a patient, and contextual information associated with the patient and representative of location and time at which the clinical and physiological measurement data were obtained; and
obtain clinician behavior data representative of behavior and activity of one or more clinicians while medically treating the patient;
one or more memory storage devices; and
one or more processors in electrical communication with the one or more memory storage devices and the communication unit, the one or more processors configured to:
determine intermittently, using an ensemble learning process, based on at least a subset of the clinical and physiological measurement data for the patient and the contextual information associated with the patient, one of a plurality of medical state prediction models best suited for a current clinical situation associated with characteristics of the patient, wherein the plurality of medical state prediction models are implemented on one or more machine learning systems;
determine, by the determined one of the plurality of medical state prediction models, based on the clinical and physiological measurement data for the patient and the clinician behavior data, prediction output data representing a medical and/or clinical state trajectory for the patient; and
provide notification output data representative of the medical and/or clinical state trajectory for the patient.

11. The system of claim 10, wherein the contextual information comprises:
treatment place at which medical treatment is being administered to the patient, and length of stay of the patient at the treatment place.

12. The system of claim 10, wherein the one or more processors configured to determine intermittently the one of the plurality of medical state prediction models are configured to:
apply at regular or irregular intervals the ensemble learning process to select at a beginning of each of the intervals, based on a state of the at least the subset of clinical and physiological measurement data and the contextual information associated with the patient at the beginning of the each of the intervals, the one of the plurality of medical state prediction models.

13. The system of claim 10, wherein the one or more processors configured to determine the prediction output data representing the medical and/or clinical state trajectory for the patient are configured to:
re-process, at the beginning of the each of the intervals, using the one of the plurality of the medical state prediction models, at least a portion of the clinical and physiological measurement data for the patient and the clinician behavior data accumulated during a specified period preceding the beginning of the each of the intervals, to determine the prediction output data representing the medical and/or clinical state trajectory for the patient during a current interval.

14. The system of claim 13, wherein the one or more processors configured to provide the notification output data are configured to:
provide notification on a user interface of a predicted medical state deterioration of the patient within a specified future time period.

15. The system of claim 14, wherein the one or more processors configured to provide the notification of the predicted medical state deterioration are configured to:
render an indicator of the predicted medical scale on a graphical severity scale to present the patient's risk severity relative to other currently hospitalized patients at a facility where the patient is hospitalized, or relative to a general population of hospitalized patients.

16. The system of claim 10, wherein the one or more processors configured to provide the notification output data are configured to:
render a list of patients being treated at a facility on the user interface and respective ones of predicted medical state deteriorations determined for the patients, the list of patients includes the patient; and
render on the user interface, in response to selecting the patient from the list of patients, a dashboard presenting information that includes the respective predicted medical state deterioration for the patient, and one or more of: details of the patient, factors that contributed to the determination of the predicted medical state, a graphic representation of the predicted medical state deterioration of the patient relative to other patients, and a trend line graph showing the predict medical state deterioration of the patient over a pre-determined period of time.

17. The system of claim 10, wherein the clinician behavior data representative of the behavior and the activity of the one or more clinicians while treating the patient comprises one or more of: frequency of surveillance by the one or more clinicians of the patient, interaction patterns between the one or more clinicians and the patient, type and frequency of clinical interventions performed by the one or more clinicians for the patient, or changes in the patient's vital signs following a clinical intervention.

18. The system of claim 10, wherein the one or more processor configured to determine the one of the plurality of medical state prediction models is configured to:
determine based further on the clinician behavior data the one of the plurality of medical state prediction models.

19. Non-transitory computer readable media comprising computer instructions executable on a processor-based device to:
obtain clinical and physiological measurement data for a patient, and contextual information associated with the patient and representative of location and time at which the clinical and physiological measurement data were obtained;
obtain clinician behavior data representative of behavior and activity of one or more clinicians while medically treating the patient;
determine intermittently, using an ensemble learning process, based on at least a subset of the clinical and physiological measurement data for the patient and the contextual information associated with the patient, one of a plurality of medical state prediction models best suited for a current clinical situation associated with characteristics of the patient, wherein the plurality of medical state prediction models are implemented on one or more machine learning systems;
determine, by the determined one of the plurality of medical state prediction models, based on the clinical and physiological measurement data for the patient and the clinician behavior data, prediction output data representing a medical and/or clinical state trajectory for the patient; and
provide notification output data representative of the medical and/or clinical state trajectory for the patient.

20. The computer readable media of claim 19, wherein the clinician behavior data representative of the behavior and the activity of the one or more clinicians while treating the patient comprises one or more of: frequency of surveillance by the one or more clinicians of the patient, interaction patterns between the one or more clinicians and the patient, type and frequency of clinical interventions performed by the one or more clinicians for the patient, or changes in the patient's vital signs following a clinical intervention.
